(19) United States
(12) Patent Application Publication
(10) Pub. No.: US 2004/0243105 A1
(43) Pub. Date: Dec. 2, 2004

(54) Autologous blood recovery apparatus

(76) Inventors: Julian Francis Ralph Swan, London (GB); Sally Louise Goodhew, Shrewsbury (GB)

Correspondence Address: DRINKER BIDDLE & REATH, ONE LOGAN SQUARE, 18TH AND CHERRY STREETS, PHILADELPHIA, PA, 19103-6996, US

(21) Appl. No.: 10/480,181

(22) PCT Filed: Jun. 6, 2002
(86) PCT No.: PCT/GB02/02406

(30) Foreign Application Priority Data
Jun. 6, 2001 (GB) 0113750.4

(51) Int. Cl. A61M 27/00

(52) U.S. Cl. 604/543

(57) ABSTRACT

The invention provides an apparatus and method to allow for the drainage of blood from the site of the wound into a blood collection device. The blood collection device is held at a vacuum as is retained within a vacuum storage chamber which is in turn conjoined to a further primary vacuum reservoir by a valve. The valve serves to control and regulate the vacuum levels between the primary vacuum reservoir and secondary chamber in which the blood collection device is contained. Following drainage of blood into the blood collection device, the flexible walls of the blood collection device allow for the optional re-transfusion of blood back into the patient from whom it was drained.

The present invention provides an apparatus for use in compensating for post operative blood loss. In particular there is provided an apparatus for use in the collection of blood from a wound drain following orthopaedic surgical procedures such as total knee replacement (TKR) and total hip replacement (THR), such that that blood can be subsequently transfused back into the patient from whom it has been collected.

The word autologous originates from Greek, with ‘auto’ meaning self and ‘logous’ meaning relation. Thus autologous can be taken as meaning ‘related to self’. Autologous blood transfusion designates the re-infusion of blood or blood components to the same individual from whom they were taken. Homologous blood is blood or blood components, from another human donor, taken and stored for later transfusion as required (Sanson 1993).

The main methods of autologous blood transfusion currently known are:

1. Pre-operative autologous donation—Collection and storage of whole blood, red blood cells, plasma and/or platelets for re-transfusion into the same patient after the operation.

2. Intra-operative autologous blood transfusion—Collection of shed blood from a wound or body cavity intra-operatively, processing, washing and re-transfusing into the same patient, immediately or later.

3. Post-operative autologous blood transfusion—Collection of blood from the wound drain for processing and re-transfusion into the same patient up to 12 hours post-operatively.

Traditionally suction and blood collection has been effected through fluid flow into bottles or rigid container devices. These have the disadvantage that the vacuum decreases within the container over time and as the container fills with fluid.

Further, such rigid collecting devices require multiple “recharges” to maintain the vacuum within them over an extended period of operation, with this vacuum being applied by either a foot pump/bellow or through a specific suction point in a hospital ward or other point of treatment.

According to the present invention there is provided an apparatus for the collection of blood from a wound, the apparatus including a first vacuum storage chamber which is connected by a valve to a second vacuum storage chamber, the second vacuum storage chamber being adapted to contain a blood collection means, the vacuum in the second vacuum storage chamber causing the blood collection means to be held at a vacuum and the blood collection means being removable from the second vacuum storage chamber.

Preferably the vacuum within the second vacuum storage chamber imparts a continues level of vacuum on the blood collection means irrespective of the amount of blood contained within the blood collecting means.

Preferably the blood collection means can be used for both the draining and re-transfusion of the blood.

Preferably the blood collection means is any form of flexible container.

Preferably the blood collection means is a bag.

Preferably the bag has a capacity of 500 ml.

Preferably the vacuum exerted on the blood collecting means by the second vacuum storage chamber is 100 mmHg.

Preferably the valve serves to maintain the vacuum level in the second vacuum storage chamber and accordingly the vacuum level in the blood collection means irrespective of the amount of blood contained within the blood collection means.

Preferably the valve further serves to maintain a vacuum in the first vacuum storage chamber during removal and fitting of the blood collection means from the second vacuum storage chamber.

Preferably the valve is a step down valve.

Alternatively the valve connecting the first and second vacuum storage chambers is a regulator, the regulator serving to keep the first vacuum storage chamber at a higher vacuum than the second vacuum storage chamber in which the blood collection means is located, such that whenever the vacuum in the second vacuum storage chamber drops below a predetermined level, the regulator can use the increased vacuum within the first vacuum storage chamber to increase the vacuum in the second vacuum storage chamber.

Preferably the first vacuum storage chamber has a vacuum of 300 mmHg.

Preferably the second vacuum storage chamber has a vacuum of 100 mmHg.

Preferably at least one vacuum gauge can be used to monitor the pressure in the first and second vacuum storage chambers.

Preferably the vacuum gauge is a rotary gauge.

Preferably the first vacuum storage chamber has sufficient capacity to impart a vacuum on the second vacuum storage chamber and thus upon the blood collection means for the duration of a complete therapeutic period, wherein the blood collection means may be changed several times.

Preferably the vacuum in the first vacuum storage chamber is imparted by a foot pump or bellow.

Alternatively the vacuum in the first vacuum storage chamber is imparted through connection to a suction port located in a hospital ward or other location of treatment.

A further aspect of the present invention provides an apparatus for effecting the collection of blood from a patient, the apparatus comprising a vacuum applying means, said vacuum applying means being adapted to support a collection means, and wherein the vacuum applying means exerts a vacuum upon said collecting means.

A further aspect of the invention provides a method of collecting blood from a patient, said method including the steps of;

Using a vacuum from a first vacuum storage chamber to establish a vacuum in a second vacuum storage chamber,

Placing a blood collection means in a second vacuum storage chamber,

imparting a vacuum on the blood collection means as a result of the vacuum which is induced in the second vacuum storage chamber,

connecting the blood collection means up to a wound drain, and

draining blood from the site of the wound.

Preferably the blood collected by means of the above-mentioned method can subsequently be re-transfused back into the patient from who the blood was originally collected.

Alternatively the blood collected by means of the above-mentioned method can subsequently be re-transfused back into a different patient from whom the blood was originally collected.

The present invention will now be described, by way of example only, with reference to the accompanying figures, wherein;

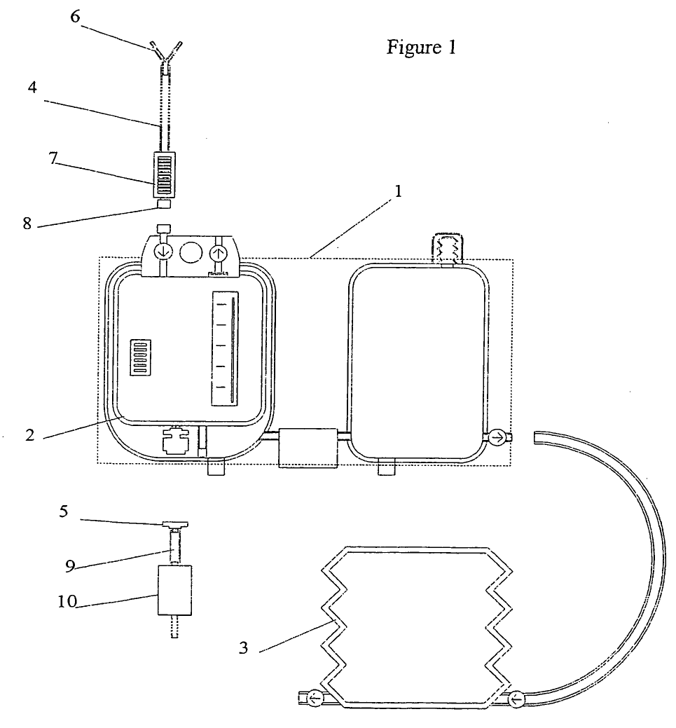

FIG. 1 shows the contributory components of the complete system and in particular the first and second vacuum storage chambers and the blood storage means being located within the second vacuum storage member,

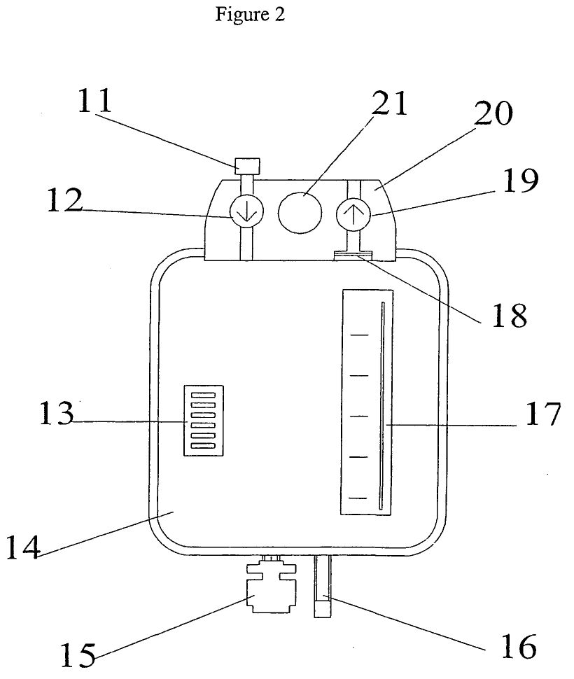

FIG. 2 shows a bag for use as a blood collection means,

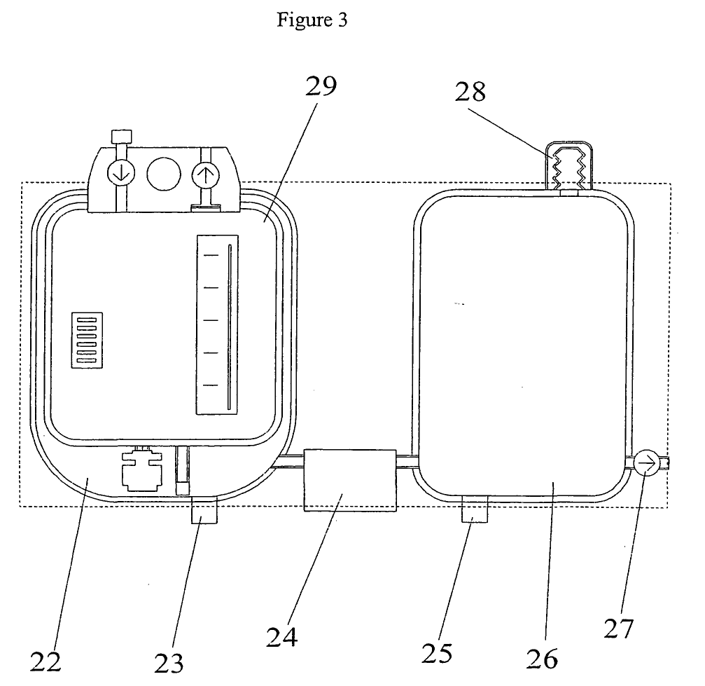

FIG. 3 shows the integration of the bag of FIG. 3 within the second vacuum storage chamber which is in turn connected to a first vacuum storage chamber by means of a regulator valve, and

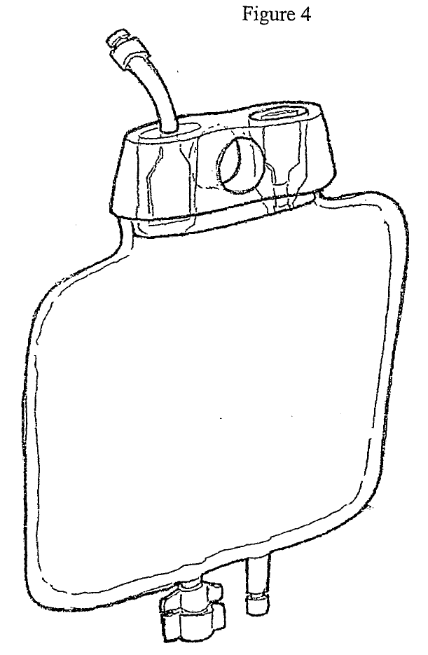

FIG. 4 is a perspective view of the bag of FIG. 3.

Referring to the drawings, FIG. 1 shows an example of a complete system of components which can contribute to the present invention.

The vacuum unit 1 consists of a first vacuum storage chamber 26 and second vacuum storage chamber 22, these chambers being best shown in FIG. 2. The first vacuum storage chamber 26 is connected to the second vacuum storage chamber 22 by means of a valve 24, which can specifically be a regulator or step down valve. The valve 24 controls the vacuum between the first and second vacuum storage chambers.

A blood collection means 2 is fitted and retained inside the second vacuum storage chamber 22. The blood collection means 2 can be any form of flexible container. The flexibility of the container allows the blood collected to be subsequently re-transfused. The blood collection means may be a bag, as shown in FIGS. 3 and 4.

Further features which can be included in the blood collection means are a dry connector 11 which allows the bag 2 (blood collection means) and the drain tube (which is connected indirectly to the side of wound drain) to be disconnected during bag changing without the risk of blood contamination or the requirement for clamps. A non return valve 12 further prevents the drained blood returning from the bag 2 towards the site of wound drain.

An electronic timer 13 allows the monitoring of the time which the blood has been stored in the bag. Regulations on the time from the initiation of collection to re-infusion of the blood mean that it is important that a bag is not used for the collection of drained blood for over four hours, and the timer allows such times to be monitored. The timer also allows predictions to be made as to the chemical state of the blood and any reactions, such as clotting which may have occurred therein.

A break seal 15 may be provided on the blood collection means 2 to provide an appropriate point for the attachment of tubing for the re-infusion of collected blood. Further, when combined with the hanging hole 21, the re-infusion procedure can be carried out using traditional apparatus. A sampling port 16 allows the blood collected within the bag 2 to be tested, while markings on the outer of the bag 2 can serve as an approximate volume scale 17.

A hydrophobic filter 18 which allows air flow there through, but which prevents blood movement can be provided, and in the example shown is integrated into an injection moulded bag top 20, which can also contain a non-return valve 19.

Other features which may be incorporated into the apparatus are shown in FIGS. 1 and 2 and include; a foot pump to produce a vacuum 3, though the vacuum may also be provided at the site of care, through for example connection to a specific port in a hospital, a drainage tube 4, a giving set 5, a Y piece for attachment to catheters 6, a timer 7, a dry connector 8, a drip chamber 9, a fine filter 10, a vacuum release valve 23 (automatic and manual activation, set to release at 100 mmHg, also activated by release of the blood collecting means from the second vacuum storage chamber), a regulator 24 (which maintains the bag cavity at a maximum of 100 mmHg), a further vacuum release valve 25 (automatic activation, set to release at 300 mmHg), and a non-return valve 27 and vacuum indicator 28.

In use, blood can drain from the site of the wound into the bag 2 (blood collection means), as a result of the vacuum imparted on the bag 2 (blood collection means) by the second vacuum storage chamber 22. The vacuum of the second vacuum storage chamber 22 is in turn a produced by the regulation of the vacuum within the first vacuum storage chamber 26 by the regulator valve 24.

A single charge of the vacuum contained within the first vacuum storage chamber 26 is sufficient to allow several bags 2 to have blood drained into them from the wound. The avoidance of the need to continually recharge the vacuum source is therefore particularly advantageous.

The valve 24 further serves to prevent loss of the vacuum contained within the first vacuum storage chamber 26 during the insertion, fixing and removal of the blood collection means 2 within the second vacuum storage chamber 22.

The valve (regulator) 24 can further be used to regulate the vacuum of the second vacuum storage chamber 22 at a constant level. This in turn ensures that the vacuum in the blood collection means 2, is maintained at a continuous level irrespective of the level of fluid which is drained in to the bag 2 (blood collection means).

The first vacuum storage chamber has a capacity of approximately 1000 ml and is primed initially to a higher vacuum than the bag cavity, e.g. 300 mmHg.

In one embodiment of the invention, the apparatus would include:

Reusable Vacuum Unit

Reusable Foot Pump

1 drain tube with Y connector

1 Re-infusion Bag

1 Giving Set and blood filter

2 drains and trocars

With the following supplementary apparatus:

1×Re-infusion Bag, 1×Infusion set with Blood filter.

Range of fg drains and trocars

The autologous transfusion of blood has several notable advantages, such as;

1. The risk of transfusion reactions is eliminated.

2. The risk of disease transmission is eliminated.

3. A source of readily available blood is available, this being particularly relevant to individuals with rare blood types.

4. The blood retains near normal levels of 2,3—DPG to provide effective tissue oxygenation.

5. The risk of alloimmunisation to red blood cells, white blood cells, platelets or plasma proteins is eliminated.

6. The risk of transfusion transmitting disease which originated from the donor is eliminated.

7. Safe transfusion is provided for patients with multiple alloantibodies or with rare blood groups.

8. Blood is available for individual patients with religious objections to allogenic transfusions.

9. The demand on the allogenic blood supply is decreased.

10. Patients are given the psychological benefit of not being exposed to the risks associated with donated blood.

11. Length of stay in hospital or place of care could be reduced.

12. The haemoglobin levels of patients receiving their own blood make a much quicker recovery than those patients receiving homogenous blood.

13. Patient acceptance—this is an acceptable alternative to Jehovah Witnesses and others with religious or moral objections to homologous transfusions.

14. Financial considerations—these being factors such as blood banks being unable to supply an adequate amount of blood, and further the cost of blood per unit, and further the cost of controlling infection as a result of rejected donor blood.

15. Reduced infection rates to wound site.

For many years blood draining into the pleural and mediastinal cavity following cardiovascular surgery has been safely re-infused without washing, therefore any operation with a large blood loss could be deemed suitable. Contra-indications and risk factors should be observed with each type of surgery.

Postoperative orthopaedic auto-transfusion systems are particularly suited for use after total knee replacement. Tourniquet inflation prevents blood loss during surgery, allowing virtually all of the blood shed to be collected postoperatively, also because the blood is released after wound wash out it is relatively free of bone chips and cement unlike that of blood from THR's. As no tourniquet is used on hip replacements (THR), alot of blood is lost during surgery hence re-transfusion drains are not commonly used on THR's.

The average postoperative blood loss for TKR's is 850 ml. Most patients received an average of 700 ml's of their own blood and did not require any further blood from blood banks. In the United Kingdom protocols generally restrict to two units being re-infused. (Carsten et al 1992) In the United States of America however there is no consensus to the amount of units re-infused, up to five units have been re-infused with no side effects. The time factor is considered to be more important. Blood collected during the first 8-10 hours is best suited for re-infusion. Large volumes of Citrate Dextrose Anti-coagulant (ACD-A) is more of a potential problem if several units are re-infused, therefore it was suggested that no anticoagulant is used after the second unit. The haemaglobin levels of patients receiving their own blood has been shown to recover quicker than those patients receiving homogenous blood.

The time from initiation of collection of the blood to transfusion must not exceed 6 hours, as recommended by the American Association of Blood Banks (AABB). Before the re-infusion starts 30 minutes have to elapse after collection. Blood collected after 12 hours must not be re-transfused, either the re-transfusion bottles can be used as an ordinary collection chamber or changed for the standard wound drains.

Blood is collected from two drains one located deep and the other superficial, which are placed within the knee cavity before closure, the tourniquet is generally released as the superficial sutures are being inserted. Where auto-transfusion drains are not used two vacuum bottles are generally used, and are changed when the bottle has either filled up or de-vacuumed. The drains are then removed after 48 hours.

400-450 mls in a reservoir that holds 500 mls or when collection time reaches 4 hours, allowing 1-2 hours to re-infuse (AABB guidelines) Larger volume reservoirs have to be monitored to ensure they do not exceed the time recommendation.

Blood becomes defribrinogenated when it comes into contact with wound tissue, this occurs in TKR's when the tourniquet is released therefore clotting in collection reservoir is unlikely. If rapid blood loss is expected after tourniquet release and defribrinogenation has not had time to occur, ACD-A can prevent a potential blood clotting problem in the reservoir. Therefore systems need to have the ability to add ACD-A. (Blood-ACD-A rate 7:1, when 40 mls of ACD-A is added a minimum of 320 ml of blood in a 500 ml reservoir is required before re-infusion is started).

Blood outside the body is susceptible to contamination caused by poor technique. Hospital protocols for the infusion of blood should be followed. Only practitioners who have been trained in the use of a specific manufacturer's apparatus should care for patients with re-transfusion drains in-situ.

Contra-indication may include:

1. Wound infection

2. Malignant lesions in the area of blood collection

3. Liver cirrhosis

4. Alcoholism

5. Shock

6. Kidney insufficiency

7. Clotting disorders

8. Haemostatics in area of wound

Possible complications may include:

Air Embolus: Air in the blood giving set can pose a life threatening complication if it enters the blood system, preventative actions would include using a blood pump with an in-line air sensor, and/or thorough training to all users on how to eliminate any air pockets when preparing the blood for re-transfusion.

Bacteremia: Re-transfusion is contra-indicated where there is wound infection (sepsis).

Haemolysis: To minimise haemolysis, vacuum should be regulated not to exceed 100 mmhg.

In one particular embodiment of the invention, blood is drained from the wound site into the bag 2 and then re-infused from the same bag. During drainage the bag is enclosed within a clam unit which is hooked onto the side of the patients bed. The clam (a variant on the system 1 detailed above) applies a constant vacuum to the bag which drains blood from the wound site.

The unit is primed using a foot pump and the vacuum stored within the first vacuum storage chamber 26 is sufficient to last for 500 ml of blood to be drained into the bag.

After 500 ml blood has been collected (or 4 hours have passed) the bag is removed from the unit, hung on a pole and infused back into the patient. Meanwhile, a further bag is loaded into the clam and the drainage procedure repeated.

Benefits of this design include the following:

1. Once the unit has been primed to the maximum, there is no need to monitor the vacuum level or re-prime throughout collection of 1 unit of blood, as the apparatus maintains vacuum at the same level, even during an increase in the level of fluid contained within the bag.

2. The foot pump allows for easy priming.

3. Manual pumping enables mobility, and frees vital wall suction.

4. Wall suction port available if required.

5. The disposable parts are minimal.

6. The dry connector between drain bag and drain tube allows for bags to be interchanged with no risk of blood contamination and no clamps.

7. Bag change over simple and quick.

8. Automatic timer, activated when drain first connected to bag.

Although the invention has been particularly shown and described with reference to the embodiment described, it will be understood by those skilled in the art that various changes in the form and details may be made without departing from the spirit and scope of the present invention.

1. An apparatus for the collection of blood from a wound, the apparatus including a first vacuum storage chamber which is connected by a valve to a second vacuum storage chamber, the second vacuum storage chamber being adapted to contain a blood collection means, the vacuum in the second vacuum storage chamber causing the blood collection means to be held at a vacuum and the blood collection means being removable from the second vacuum storage chamber.

2. Apparatus as claimed in claim 1, wherein the valve maintains the vacuum in the second vacuum storage chamber and accordingly the vacuum imparted on the blood collection means irrespective of the amount of blood contained within the blood collection means.

3. Apparatus as claimed in claim 1, wherein the valve further maintains a vacuum in the first vacuum storage chamber during removal and fitting of the blood collection means from the second vacuum storage chamber.

4. Apparatus as claimed in claim 1 wherein the valve is a step down valve.

5. Apparatus as claimed in claim 1 wherein the valve is a regulator which maintains the vacuum level in the second vacuum storage chamber using the vacuum stored in the first vacuum storage chamber.

6. Apparatus as claimed in claim 1 wherein the first vacuum storage chamber has a vacuum of 300 mmHg.

7. Apparatus as claimed in claim 1 wherein the blood collection means is any form of flexible container.

8. Apparatus as claimed in claim 1 wherein the blood collection means is a bag.

9. Apparatus as claimed in claim 8 wherein the bag has a capacity of 500 ml.

10. Apparatus as claimed in claim 1 wherein the vacuum exerted on the blood collecting means by the second vacuum storage chamber is 100 mmHg.

11. Apparatus as claimed in claim 1 wherein at least one pressure gauge is used to monitor the pressure in the first and second vacuum storage chambers.

12. Apparatus as claimed claim 1 wherein the blood collection means can be used in the re-transfusion of the blood collected therein.

13. A method of collecting blood from a patient, said method including the steps of;
using a vacuum from a first vacuum storage chamber to establish a vacuum in a second vacuum storage chamber,
placing a blood collection means in a second vacuum storage chamber,
imparting a vacuum on the blood collection means as a result of the vacuum which is induced in the second vacuum storage chamber,
connecting the blood collection means up to a wound drain, and
draining blood from the site of the wound.

14. A method as claimed in claim 13, wherein the blood collected in the blood collection means can be subsequently re-infused into a patient.
